(19) United States
(12) Patent Application Publication
(10) Pub. No.: US 2022/0334099 A1
(43) Pub. Date: Oct. 20, 2022

(54) BLOOD ANALYSIS APPARATUS, BLOOD ANALYSIS METHOD, AND STORAGE MEDIUM

(71) Applicant: Shenzhen Mindray Bio-Medical Electronics Co., Ltd., Shenzhen (CN)

(72) Inventors: Wenbo ZHENG, Shenzhen (CN); Bo YE, Shenzhen (CN); Donglan YAO, Shenzhen (CN); Yi YE, Shenzhen (CN); Huan QI, Shenzhen (CN)

(21) Appl. No.: 17/720,018

(22) Filed: Apr. 13, 2022

(30) Foreign Application Priority Data
Apr. 14, 2021 (CN) 202110400024.6

(51) Int. Cl. G01N 33/49; G01N 21/64; G01N 21/51

(52) U.S. Cl. CPC G01N 33/49; G01N 21/6486; G01N 21/6428; G01N 21/51; G01N 2021/4707

(57) ABSTRACT

Blood analysis apparatus, method, and storage medium are provided, including: obtaining optical signals of a first test sample; determining a first test result of a blood sample from the optical signals of the first test sample; determining whether any one or a combination of reticulocytes, immature platelets and large-volume platelets in the blood sample is abnormal from the optical signals of the first test sample; outputting the first test result if it is determined that reticulocytes, immature platelets and large-volume platelets in the blood sample are normal; if it is determined that any one or a combination of reticulocytes, immature platelets and large-volume platelets in the blood sample is abnormal, preparing a second test sample and obtaining optical signals of the second test sample; and obtaining a second test result of the blood sample from the optical signals of the second test sample and outputting the first test result.

### CROSS-REFERENCE TO RELATED APPLICATIONS

This application claims priority to Chinese Patent Application No. 202110400024.6, entitled “BLOOD ANALYSIS APPARATUS, BLOOD ANALYSIS METHOD, AND STORAGE MEDIUM,” and filed on Apr. 14, 2021, the contents of which are incorporated herein by reference.

### TECHNICAL FIELD

The disclosure relates to the technical field of in vitro testing, and in particular, to a blood analysis apparatus, a blood analysis method, and a storage medium.

### BACKGROUND

Blood analysis is widely used in medical research and testing to obtain information about blood cells such as reticulocytes, immature platelets, and large-volume platelets. Common blood cell analyzers usually use an independent reticulocyte test channel and reagent to detect information about reticulocytes, immature platelets, and large-volume platelets.

At present, the independent reticulocyte test channel is usually used only for special patients. If the reticulocyte test channel needs to be used for ordinary patients, the reticulocyte test channel needs to be kept open normally, which requires consumption of more reagents and increase in an operating load of an instrument, thus increasing the instrument cost and the reagent cost of the blood cell analyzers.

### SUMMARY

In view of this, embodiments of the disclosure provide a blood analysis apparatus, a blood analysis method, and a storage medium, to reduce the instrument cost and the reagent cost of a blood cell analyzer.

To achieve the foregoing objective, embodiments of the disclosure provide the following technical solutions.

According to a first aspect, in an embodiment, a blood analysis apparatus is provided, the apparatus including:

a blood sample supply portion configured to provide a blood sample;

a reagent supply portion configured to provide a reaction reagent;

at least one mixing chamber configured to receive the blood sample provided by the blood sample supply portion and the reaction reagent provided by the reagent supply portion to prepare a test sample;

a measuring portion comprising an optical detection portion, wherein the optical detection portion comprises a flow chamber, a light source, and an optical detector; wherein the flow chamber communicates with the mixing chamber to allow cells of the test sample to pass therethrough one by one, the light source is configured to irradiate cells passing through the flow chamber, and the optical detector is configured to obtain optical signals of the cells passing through the flow chamber; and

a processor, wherein

the processor controls the blood sample supply portion to provide the blood sample to the mixing chamber, and controls the reagent supply portion to provide a first reaction reagent to the mixing chamber to prepare a first test sample in the mixing chamber, wherein the first reaction reagent contains a hemolytic agent; the processor further controls the optical detection portion to obtain optical signals of the first test sample, and determines a first test result of the blood sample from the optical signals of the first test sample, wherein the first test result of the blood sample comprises a white blood cell count result and/or a white blood cell classification result;

the processor further determines whether any one or a combination of reticulocytes, immature platelets and large-volume platelets in the blood sample is abnormal from the optical signals of the first test sample;

the processor further outputs the first test result if it is determined that the reticulocytes, the immature platelets and the large-volume platelets in the blood sample are all normal;

if it is determined that any one or a combination of the reticulocytes, the immature platelets and the large-volume platelets in the blood sample is abnormal, the processor further controls the blood sample supply portion to provide the blood sample to the mixing chamber, and controls the reagent supply portion to provide a second reaction reagent to the mixing chamber to prepare a second test sample in the mixing chamber, wherein the second reaction reagent contains no hemolytic agent; and the processor further controls the optical detection portion to obtain optical signals of the second test sample, obtains a second test result of the blood sample from the optical signals of the second test sample, and outputs the first test result, wherein the second test result comprises any one or a combination of a reticulocyte parameter, an immature platelet parameter and a large-volume platelet parameter of the second test sample.

According to a second aspect, in an embodiment, a blood analysis apparatus is provided, the apparatus including:

a blood sample supply portion configured to provide a blood sample;

a reagent supply portion configured to provide a reaction reagent;

at least one mixing chamber configured to receive the blood sample provided by the blood sample supply portion and the reaction reagent provided by the reagent supply portion to prepare a test sample;

a measuring portion comprising an optical detection portion, wherein the optical detection portion comprises a flow chamber, a light source, and an optical detector; wherein the flow chamber communicates with the mixing chamber to allow cells of the test sample to pass therethrough one by one, the light source is configured to irradiate cells passing through the flow chamber, and the optical detector is configured to obtain optical signals of the cells passing through the flow chamber; and

the blood analysis apparatus further comprises a first measurement mode and a second measurement mode, and the blood analysis apparatus further comprises a processor, wherein

the processor determines whether the second measurement mode is enabled, if the second measurement mode is enabled, the processor controls the blood sample supply portion to provide the blood sample to the mixing chamber, and controls the reagent supply portion to provide a second reaction reagent to the mixing chamber to prepare a second test sample in the mixing chamber, wherein the second reaction reagent contains no hemolytic agent; the processor further controls the optical detection portion to obtain optical signals of the second test sample and obtains a second test result of the blood sample from the optical signals of the second test sample, wherein the second test result comprises any one or a combination of a reticulocyte parameter, an immature platelet parameter and a large-volume platelet parameter of the second test sample; or

if the second measurement mode is not enabled, in the first measurement mode, the processor controls the blood sample supply portion to provide the blood sample to the mixing chamber, and controls the reagent supply portion to provide a first reaction reagent to the mixing chamber to prepare a first test sample in the mixing chamber, wherein the first reaction reagent contains a hemolytic agent; the processor further controls the optical detection portion to obtain optical signals of the first test sample, and determines a first test result of the blood sample from the optical signals of the first test sample, wherein the first test result of the blood sample comprises a white blood cell count result and/or a white blood cell classification result; wherein the first test sample is prepared from the blood sample provided by the blood supply portion and at least the first reaction reagent provided by the reagent supply portion, which are received by the mixing chamber; the processor further determines whether to enable the second measurement mode by determining whether any one or a combination of reticulocytes, immature platelets and large-volume platelets in the blood sample is abnormal from the optical signals of the first test sample; and if it is determined that any one or a combination of the reticulocytes, the immature platelets and the large-volume platelets in the blood sample is abnormal, it is determined that the second measurement mode should be enabled and the first test result is outputted; if it is determined that the reticulocytes, the immature platelets and the large-volume platelets in the blood sample are all normal, it is determined that the second measurement mode should not be enabled and the first test result is outputted.

In an embodiment, the optical signals of the first test sample include forward-scattered light signals and fluorescence signals, wherein the processor further controls the blood sample supply portion to provide the blood sample to the mixing chamber and controls the reagent supply portion to provide the hemolytic agent and a first fluorescent agent to the mixing chamber to prepare the first test sample in the mixing chamber.

In an embodiment, the processor determining whether any one or a combination of the reticulocytes, the immature platelets and the large-volume platelets in the blood sample is abnormal from the optical signals of the first test sample includes:

the processor generating a scatter diagram corresponding to the first test sample from the optical signals of the first test sample;

the processor obtaining, on the scatter diagram of the first test sample, feature regions corresponding to the reticulocytes, the immature platelets and the large-volume platelets in the blood sample; and

the processor determining whether any one or a combination of the reticulocytes, the immature platelets and the large-volume platelets in the blood sample is abnormal based on scatter point information in the obtained feature regions.

In an embodiment, the processor determining whether any one or a combination of the reticulocytes, the immature platelets and the large-volume platelets in the blood sample is abnormal based on scatter point information in the obtained feature regions includes:

the processor counting first scatter points in the feature region corresponding to the reticulocytes in the blood sample;

the processor determining that the reticulocytes in the blood sample are normal if the count of the first scatter points is within a first threshold range;

the processor determining that the reticulocytes in the blood sample are abnormal if the count of the first scatter points is outside the first threshold range;

the processor counting second scatter points in the feature region corresponding to the immature platelets in the blood sample;

the processor determining that the immature platelets in the blood sample are normal if the count of the second scatter points is within a second threshold range;

the processor determining that the immature platelets in the blood sample are abnormal if the count of the second scatter points is outside the second threshold range; the processor counting third scatter points in the feature region corresponding to the large-volume platelets in the blood sample;

the processor determining that the large-volume platelets in the blood sample are normal if the count of the third scatter points is within a third threshold range;

the processor determining that the large-volume platelets in the blood sample are abnormal if the count of the third scatter points is outside the third threshold range.

In an embodiment, the processor further generates a corresponding alarm prompt if it is determined that any one or a combination of the reticulocytes, the immature platelets and the large-volume platelets in the blood sample is abnormal.

In an embodiment, the optical signals of the second test sample include forward-scattered light signals and fluorescence signals, wherein the processor further controls the blood sample supply portion to provide the blood sample to the mixing chamber and controls the reagent supply portion to provide a diluent and a second fluorescent agent to the mixing chamber to prepare the second test sample in the mixing chamber.

According to a third aspect, in an embodiment, a blood analysis method is provided, the method including:

obtaining optical signals of a first test sample, wherein the first test sample is obtained by treating a blood sample with at least a first reaction reagent, and the first reaction reagent contains a hemolytic agent;

determining a first test result of the blood sample from the optical signals of the first test sample, wherein the first test result comprises a white blood cell count result and/or a white blood cell classification result;

determining whether any one or a combination of reticulocytes, immature platelets and large-volume platelets in the blood sample is abnormal from the optical signals of the first test sample;

outputting the first test result if it is determined that the reticulocytes, the immature platelets and the large-volume platelets in the blood sample are all normal;

if it is determined that any one or a combination of the reticulocytes, the immature platelets and the large-volume platelets in the blood sample is abnormal, preparing a second test sample, and obtaining optical signals of the second test sample, wherein the second test sample is obtained by treating the blood sample with at least a second reaction reagent, and the second reaction reagent contains no hemolytic agent; and

obtaining a second test result of the blood sample from the optical signals of the second test sample, and outputting the first test result, wherein the second test result comprises any one or a combination of a reticulocyte parameter, an immature platelet parameter and a large-volume platelet parameter of the second test sample.

According to a fourth aspect, in an embodiment, a blood analysis method is provided, the method including:

determining whether a second measurement mode is enabled, wherein a blood analysis apparatus comprises a first measurement mode and a second measurement mode;

if the second measurement mode is enabled, preparing a second test sample, obtaining optical signals of the second test sample, and obtaining a second test result of a blood sample from the optical signals of the second test sample, wherein the second test result comprises any one or a combination of a reticulocyte parameter, an immature platelet parameter and a large-volume platelet parameter of the second test sample, the second test sample is obtained by treating the blood sample with at least a second reaction reagent, and the second reaction reagent contains no hemolytic agent;

if the second measurement mode is not enabled, obtaining optical signals of the first test sample in the first measurement mode, wherein the optical signals of the first test sample are used to determine a first test result of the blood sample, the first test result comprises a white blood cell count result and/or a white blood cell classification result, the first test sample is obtained by treating the blood sample with at least a first reaction reagent, and the first reaction reagent contains a hemolytic agent;

determining whether to enable the second measurement mode by determining whether any one or a combination of reticulocytes, immature platelets and large-volume platelets in the blood sample is abnormal from the optical signals of the first test sample; and if it is determined that any one or a combination of the reticulocytes, the immature platelets and the large-volume platelets in the blood sample is abnormal, it is determined that the second measurement mode should be enabled and the first test result is output; and if it is determined that the reticulocytes, the immature platelets and the large-volume platelets in the blood sample each are normal, it is determined that the second measurement mode should not be enabled and the first test result is output.

In an embodiment, the first test sample is obtained by treating the blood sample with the hemolytic agent and a first fluorescent agent, and the optical signals of the first test sample include forward-scattered light signals and fluorescence signals.

In an embodiment, determining whether any one or a combination of the reticulocytes, the immature platelets and the large-volume platelets in the blood sample is abnormal from the optical signals of the first test sample includes:

generating a scatter diagram corresponding to the first test sample from the optical signal of the first test sample;

obtaining, on the scatter diagram of the first test sample, feature regions corresponding to the reticulocytes, the immature platelets and the large-volume platelets in the blood sample; and

determining whether any one or a combination of the reticulocytes, the immature platelets and the large-volume platelets in the blood sample is abnormal based on scatter point information in the obtained feature regions.

In an embodiment, determining whether any one or a combination of the reticulocytes, the immature platelets and the large-volume platelets in the blood sample is abnormal based on scatter point information in the obtained feature regions includes:

counting first scatter points in the feature region corresponding to the reticulocytes in the blood sample;

determining that the reticulocytes in the blood sample are normal if the count of the first scatter points is within a first threshold range;

determining that the reticulocytes in the blood sample are abnormal if the count of the first scatter points is outside the first threshold range;

counting second scatter points in the feature region corresponding to the immature platelets in the blood sample;

determining that the immature platelets in the blood sample are normal if the count of the second scatter points is within a second threshold range; or determining that the immature platelets in the blood sample are abnormal if the count of the second scatter points is outside the second threshold range;

counting third scatter points in the feature region corresponding to the large-volume platelets in the blood sample; and

determining that the large-volume platelets in the blood sample are normal if the count of the third scatter points is within a third threshold range; or

determining that the large-volume platelets in the blood sample are abnormal if the count of the third scatter points is outside the third threshold range.

In an embodiment, the method further includes: generating a corresponding alarm prompt if it is determined that any one or a combination of the reticulocytes, the immature platelets and the large-volume platelets in the blood sample is abnormal.

In an embodiment, the second test sample is obtained by treating the blood sample with a diluent and a second fluorescent agent, and the optical signals of the second test sample include forward-scattered light signals and fluorescence signals.

According to a fifth aspect, in an embodiment, a computer-readable storage medium is provided, including a program, where the program is executable by a processor to implement a blood analysis method of any one of the embodiments herein.

As can be seen from the foregoing technical solutions, the embodiments of the disclosure have the following advantages:

in the embodiments of the disclosure, whether to prepare the second test sample is determined depending on the optical signals of the first test sample, and it is not required to keep the reticulocyte test channel open normally, which reduces the instrument cost and the reagent cost of a blood cell analyzer.

### BRIEF DESCRIPTION OF THE DRAWINGS

In order to more clearly illustrate technical solutions in the embodiments of the disclosure or in the prior art, a brief introduction to the drawings will be provided below. The drawings in the following description illustrate only some of the embodiments of the disclosure, and those of ordinary skill in the art would also be able to obtain other drawings from these drawings without any creative effort.

FIG. 1 is a structural schematic diagram of a blood analysis apparatus provided in an embodiment of the disclosure;

FIG. 2 is a structural schematic diagram of an optical detection portion provided in an embodiment of the disclosure;

FIG. 3 is another structural schematic diagram of the optical detection portion provided in the embodiment of the disclosure;

FIG. 4 is still another structural schematic diagram of the optical detection portion provided in the embodiment of the disclosure;

FIG. 5 is a scatter diagram of a first test sample generated on the basis of forward-scattered light signals and fluorescence signals provided in an embodiment of the disclosure;

FIG. 6A is an example of a scatter diagram generated on the basis of forward-scattered light signals and side-scattered light signals of a first test sample prepared from a normal blood sample;

FIG. 6B is an example of a scatter diagram generated on the basis of forward-scattered light signals and fluorescence signals of a test sample prepared from a normal blood sample;

FIG. 6C is an example of a scatter diagram generated when a reticulocyte test is performed on a normal blood sample;

FIG. 7A is an example of a scatter diagram generated on the basis of forward-scattered light signals and side-scattered light signals of a first test sample prepared from a blood sample with a high reticulocyte percentage;

FIG. 7B is an example of a scatter diagram generated on the basis of forward-scattered light signals and fluorescence signals of a test sample prepared from a blood sample with a high reticulocyte percentage;

FIG. 7C is an example of a scatter diagram generated when a reticulocyte test is performed on a blood sample with a high reticulocyte percentage;

FIG. 8A is an example of a scatter diagram generated on the basis of forward-scattered light signals and side-scattered light signals of a first test sample prepared from a normal blood sample;

FIG. 8B is an example of a scatter diagram generated on the basis of forward-scattered light signals and fluorescence signals of a test sample prepared from a normal blood sample;

FIG. 8C is an example of a scatter diagram generated when an immature-platelet test is performed on a normal blood sample;

FIG. 9A is an example of a scatter diagram generated on the basis of forward-scattered light signals and side-scattered light signals of a first test sample and prepared from a blood sample with a high immature platelet percentage;

FIG. 9B is an example of a scatter diagram generated on the basis of forward-scattered light signals and fluorescence signals of a test prepared from a blood sample with a high immature platelet percentage;

FIG. 9C is an example of a scatter diagram generated when an immature-platelet test is performed on a blood sample with a high immature platelet percentage;

FIG. 10A is an example of a scatter diagram generated when a large-volume platelet test is performed on a normal blood sample;

FIG. 10B is an example of a scatter diagram generated when a large-volume platelet test is performed on a blood sample with a large-volume platelet percentage;

FIG. 11 is a table diagram for illustrating some test modes of a BC-6800 blood cell analyzer, a BC-6000 blood cell analyzer and a BC-6800Plus blood cell analyzer from Shenzhen Mindray Bio-medical Electronics Co., Ltd. provided in an embodiment of the disclosure;

FIG. 12 is a flowchart of a blood analysis method provided in an embodiment of the disclosure;

FIG. 13 is a flowchart for determining whether any one or a combination of reticulocytes, immature platelets and large-volume platelets in a blood sample is abnormal as provided in an embodiment of the disclosure; and

FIG. 14 is another flowchart of a blood analysis method provided in an embodiment of the disclosure.

### DESCRIPTION OF THE PREFERRED EMBODIMENTS

The technical solutions in the embodiments of the disclosure will be described below clearly and completely in conjunction with the drawings. The embodiments described are merely some of, rather than all of the embodiments of the disclosure.

Based on the embodiments given in the disclosure, all other embodiments that would be obtained by those of ordinary skill in the art without involving any creative effort shall all fall within the scope of protection of the disclosure.

In the disclosure, the terms “comprise”, “include” or any variation thereof are intended to cover a non-exclusive inclusion, so that a process, method, article or device that includes a series of elements not only includes those elements but also includes other elements not expressly listed or further includes elements inherent to such a process, method, article, or device. In the absence of more restrictions, the element defined by the phrase “including a/an . . . ” does not exclude the presence of an additional identical element in the process, method, article or device that includes the element.

A reticulocyte (RET) is a transitional cell between an orthochromatic erythrocyte and a mature erythrocyte. Reticulocyte count is an important indicator for determining erythropoiesis of bone marrow and observing a curative effect.

Reticulated platelets (RPs), that is, immature platelets, represent newly generated platelets in human peripheral blood released from bone marrow megakaryocytes. The percentage of RP and its absolute value count are of great significance to kinetics of platelet formation and mechanism of thrombocytopenia. RP count reflects a rate of platelet formation, and especially a ratio of the number of RPs to the total number of platelets (immature platelet fraction, IPF) can more accurately reflect the rate of platelet formation.

For the detection of reticulocytes, immature platelets and large-volume platelets (large PLTs for short), current mainstream blood cell analyzers in the industry use an independent channel and reagent for detection, which increases the instrument cost and the reagent cost. For example, patent U.S. Pat. No. 7,892,850 provides a method for detecting immature platelets, in which an independent reagent system and detection apparatus are used to classify and count immature platelets.

It may be understood that the counting of reticulocytes, immature platelets and large-volume platelets is of great significance for the diagnosis and treatment of some special diseases. However, the counting of reticulocytes, immature platelets and large-volume platelets are obtained in an independent channel by using a special reagent. However, such an independent channel, or the detection of reticulocytes, immature platelets and large-volume platelets, is not necessary for most ordinary patients, which leads in a contradiction.

The inventors have found through research that for the analysis of blood samples, related tests on white blood cells, such as white blood cell count and white blood cell classification, are generally performed. White blood cell count refers to counting all white blood cells in a blood sample. White blood cell classification refers to classifying white blood cells into different types.

White blood cell count or white blood cell classification is a common test item in blood sample analysis. Both white blood cell count and white blood cell classification may be measured by using laser scattering principle. Specifically, cells are irradiated by laser light, and tests on white blood cells such as counting and classification are completed by collecting optical signals (such as forward-scattered light signals, side-scattered light signals and even fluorescence signals) generated after the cells are irradiated and analyzing the optical signals.

Through repeated experiments, the inventors have found that in the tests on white blood cells such as count or classification by using laser scattering principle, the collected optical signals may also be used for an initial test on any one or a combination of reticulocytes, immature platelets and large-volume platelets, that is, to determine whether any one or a combination of reticulocytes, immature platelets and large-volume platelets in a blood sample is abnormal. If any one or a combination of the reticulocytes, the immature platelets and the large-volume platelets in the blood sample is abnormal, then any one or a combination of a reticulocyte parameter, an immature platelet parameter and a large-volume platelet parameter is further detected through an independent channel for reticulocytes for example, to obtain a more accurate test result; and if the reticulocytes, the immature platelets and the large-volume platelets in the blood sample are all normal, it means that there is no need to start an independent channel for reticulocytes for further testing.

By application of some embodiments of the disclosure, an actual application scenario will be provided: some routine detection modes are enabled or some routine items are tested for patients, such as white blood cell count and white blood cell classification. Based on optical signals collected from these items, it is determined whether it is necessary to count any one or a combination of reticulocytes, immature platelets and large-volume platelets, so as to reduce the instrument cost and the reagent cost of a blood cell analyzer.

The disclosure will be further described in detail below in conjunction with the contents of various embodiments.

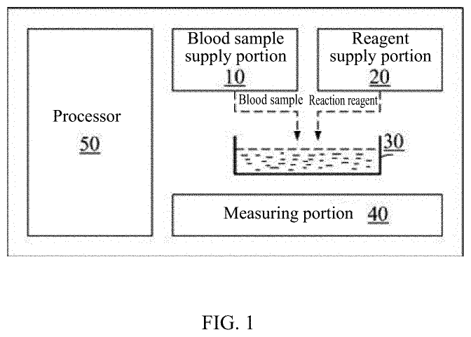

A blood analysis apparatus is disclosed in some embodiments of the disclosure. Referring to FIG. 1, a blood analysis apparatus in some embodiments may include a blood sample supply portion 10, a reagent supply portion 20, a mixing chamber 30, a measuring portion 40, and a processor 50. Specifically, the blood sample supply portion 10 is configured to provide a blood sample; the reagent supply portion 20 is configured to provide a reaction reagent; the mixing chamber 30 is configured to receive the blood sample provided by the blood sample supply portion 10 and the reaction reagent provided by the reagent supply portion 20 to prepare a test sample; and the measuring portion 40 is configured to test the prepared test sample. See the following description for details.

In some embodiments, the blood sample supply portion 10 may include a sample needle which performs a two-dimensional or three-dimensional movement in space by using a two-dimensional or three-dimensional driving mechanism, such that the sample needle may move to aspirate a blood sample in a container (such as a sample tube) carrying the blood sample, and then move to, for example, a mixing chamber 30, which is configured to provide a reaction place for the blood sample to be tested and reagents, and discharge the blood sample to the mixing chamber 30. In some embodiments, the reagent supply portion 20 may include a reagent tray and a reagent needle. The reagent tray is configured as a disk-like structure and has a plurality of positions for carrying reagent containers. The reagent tray can rotate and drive the reagent containers carried thereby to rotate, so as to rotate the reagent containers to specific positions, such as a position at which the reagent needle aspirates the reagent; and the reagent needle can perform a two-dimensional or three-dimensional movement in space by using a two-dimensional or three-dimensional driving mechanism, such that the reagent needle can move to aspirate the reaction reagent carried by the reagent tray and discharge the reaction reagent into the mixing chamber 30. In some other embodiments, the reagent supply portion 20 may also include a region for carrying reaction reagents and a reagent needle, where the reaction reagents are fixedly placed, and the reagent needle moves to aspirate different reaction reagents and discharge the reaction reagents to the mixing chamber 30.

There may be one or more mixing chambers 30. The one or more mixing chambers 30 are configured to provide a treatment place or reaction place for the blood sample and the reaction reagents. A same mixing chamber 30 may be shared for different test items; or different mixing chambers 30 may be used for different test items, for example, one mixing chamber 30 may be used for a white blood cell classification test item, and another mixing chamber 30 may be used for a test item of any one or a combination of reticulocyte, immature platelets and large-volume platelets.

A test sample may be obtained by treating a blood sample with a reaction reagent. In some embodiments, the reaction reagent includes any one or a combination of a hemolytic agent, a fluorescent agent, and a diluent. The hemolytic agent is a reagent that can dissolve red blood cells in a blood sample. Specifically, the hemolytic agent may be any one or a combination of a cationic surfactant, a non-ionic surfactant, an anionic surfactant and an amphiphilic surfactant. The diluent is used to dilute a blood sample. The fluorescent agent is used to stain a blood sample. For example, the fluorescent agent may be pyronine, acridine orange, thiazole orange, or the like.

The measuring portion 40 is configured to test the prepared test sample. In some embodiments, the measuring portion 40 may include an optical detection portion 60, and the optical detection portion 60 can measure the blood sample by using the laser scattering principle. The principle is as follows: cells are irradiated by laser light, and the cells can be classified and counted by collecting optical signals generated after the cells are irradiated, such as scattered light and fluorescence. Of course, in some embodiments, if the cells are not treated with the fluorescent agent, inherently no fluorescence can be collected. Details of the optical detection portion 60 in the measuring portion 40 will be described below.

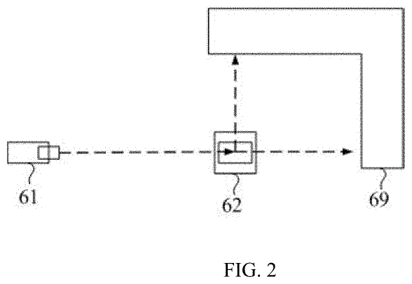

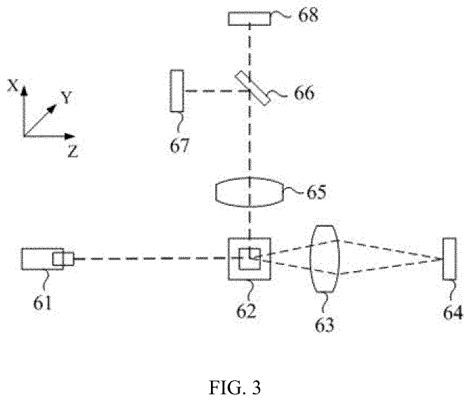

Referring to FIG. 2, the optical detection portion 60 may include a light source 61, a flow chamber 62 and an optical detector 69. The flow chamber 62 communicates with the mixing chamber 30 and is configured to allow cells of the test sample to pass therethrough one by one. The light source 61 is configured to irradiate the cells passing through the flow chamber 62, and the optical detector 69 is configured to obtain optical signals of the cells passing through the flow chamber 62. FIG. 3 shows a specific example of the optical detection portion 60. The optical detector 69 may include a lens group 63 for collecting forward-scattered light, a photoelectric detector 64 for converting the collected forward-scattered light from optical signals to electrical signals, a lens group 65 for collecting side-scattered light and side fluorescence, a dichroscope 66, a photoelectric detector 67 for converting the collected side-scattered light from optical signals to electrical signals, and a photoelectric detector 68 for converting the collected side fluorescence from optical signals to electrical signals. The dichroscope 66 is configured to split light, and divides the mixed side-scattered light and side fluorescence into two paths, one path is the side-scattered light, and the other path is the side fluorescence. It should be noted that the optical signals herein may be either optical signals or electrical signals obtained by conversion from the optical signals, the optical signals and electrical signals are essentially the same in information contained in representing a cell test result.

The structure of the optical detection portion 60 shown in FIG. 3 is used as an example to explain how the optical detection portion 60 obtains optical signals of a test sample.

The flow chamber 62 is configured to allow the cells of the test sample to pass therethrough one by one. For example, in the mixing chamber 30, red blood cells in the blood sample are dissolved by some reaction reagents such as a hemolytic agent, or further stained by a fluorescent agent, and then a sheath flow technology is adopted, such that the cells in the prepared test sample successively pass through the flow chamber 62 one by one. A Y-axis direction in the figure refers to a direction perpendicular to the paper. The light source 61 is configured to irradiate the cells passing through the flow chamber 62. In some embodiments, the light source 61 is a laser, such as a helium-neon laser or a semiconductor laser. The light from the light source 61 will be scattered all around when irradiating the cells in the flow chamber 62. Therefore, when the cells in the prepared test sample pass through the flow chamber 62 one by one by under the action of a sheath flow, the light emitted by the light source 61 irradiates the cells passing through the flow chamber 62, and the light irradiated on the cells will be scattered all around. Forward-scattered light (for example, in a Z-axis direction in FIG. 3) is collected by the lens group 63 to make the light reach the photoelectric detector 64, such that the processor 50 can obtain forward-scattered light information of the cells from the photoelectric detector 64. In addition, side light (such as in an X-axis direction in FIG. 3) is collected by the lens group 65 in a direction perpendicular to the light irradiated on the cells, and the collected side light is reflected and refracted by the dichroscope 66. Side-scattered light of the side light is reflected when passing through the dichroscope 66, and then reaches the corresponding photoelectric detector 67, and side fluorescence of the side light is refracted or transmitted and then also reaches the corresponding photoelectric detector 68, such that the processor 50 can obtain side-scattered light information of the cells from the photoelectric detector 67 and obtain side fluorescence information of the cells from the photoelectric detector 68.

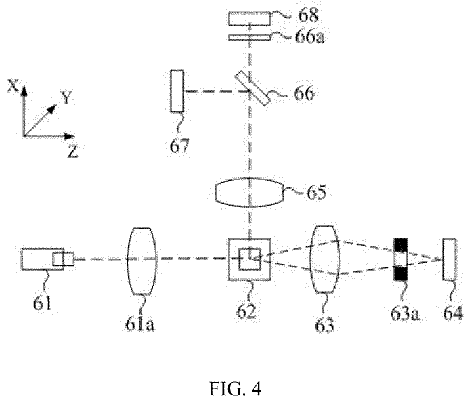

Referring to FIG. 4, another example of the optical detection portion 60 is shown. To make the performance of the light from the light source 61 irradiated on the flow chamber 62 better, a collimating lens 61a may be introduced between the light source 61 and the flow chamber 62. The light emitted by the light source 61 is collimated by the collimating lens 61a and then is irradiated on the cells passing through the flow chamber 62. In some examples, to reduce the noise in the collected fluorescence (that is, no interference from other light), an optical filter 66a may be further provided in front of the photoelectric detector 68, and the side-fluorescence after splitting by the dichroscope 66 will pass through the optical filter 66a before reaching the photoelectric detector 68. In some embodiments, after the lens group 63 collects the forward-scattered light, a diaphragm 63a is further introduced to limit an angle of the forward-scattered light that finally reaches the photoelectric detector 64, for example, the forward-scattered light is limited to low-angle (or small-angle) forward-scattered light.

It should be noted that the processor 50 in some embodiments of the disclosure includes, but is not limited to, a central processing unit (CPU), a micro controller unit (MCU), a field-programmable gate array (FPGA), a digital signal processor (DSP) and other apparatuses for analyzing computer instructions and processing data in computer software. In some embodiments, the processor 50 is configured to execute each computer application program in a non-transitory computer-readable storage medium, such that the blood analysis apparatus carries out a corresponding test flow and analyzes and processes optical signals detected by the optical detector 69, so as to obtain a corresponding test result.

In some embodiments of the disclosure, the processor 50 controls the blood sample supply portion 10 to provide the blood sample to the mixing chamber 30 and controls the reagent supply portion 20 to provide a first reaction reagent to the mixing chamber 30 to prepare a first test sample in the mixing chamber, that is, the first test sample is obtained by treating the blood sample with at least the first reaction reagent, and the first reaction reagent contains a hemolytic agent. The processor 50 controls the optical detection portion 69 to obtain optical signals of the first test sample, and determines a first test result of the blood sample from the optical signals of the first test sample, wherein the first test result includes a white blood cell count result and/or a white blood cell classification result.

The processor 50 further determines whether any one or a combination of reticulocytes, immature platelets and large-volume platelets in the blood sample is abnormal from the optical signals of the first test sample. The processor 50 outputs the first test result if it is determined that the reticulocytes, the immature platelets and the large-volume platelets in the blood sample are all normal. If it is determined that any one or a combination of the reticulocytes, the immature platelets and the large-volume platelets in the blood sample is abnormal, the processor 50 controls the blood sample supply portion 10 to provide the blood sample to the mixing chamber 30, and controls the reagent supply portion 20 to provide a second reaction reagent to the mixing chamber 30 to prepare a second test sample in the mixing chamber, wherein the second reaction reagent contains no hemolytic agent, that is, the second test sample is obtained by treating the blood sample with at least the second reaction reagent. The processor 50 controls the optical detection portion 69 to obtain optical signals of the second test sample, obtains a second test result of the blood sample from the optical signals of the second test sample, and outputs the first test result, wherein the second test result includes any one or a combination of a reticulocyte parameter, an immature platelet parameter and a large-volume platelet parameter of the second test sample.

That is, if it is determined that any one or a combination of the reticulocytes, the immature platelets and the large-volume platelets in the blood sample is abnormal by using the optical signals of the first test sample, the second test sample is prepared, and after the second test result of the blood sample is obtained from the optical signals of the second test sample, the obtained first test result is further output.

It should be noted that the reticulocyte parameter of the second test sample mentioned above specifically refers to a parameter that can reflect the number of reticulocytes, such as the count or proportion of reticulocytes, similarly, the immature platelet parameter of the second test sample specifically refers to a parameter that can reflect the number of immature platelets, such as the count or proportion (for example, IRF) of immature platelets, and the large-volume platelet parameter of the second test sample specifically refers to a parameter that can reflect the number of large-volume platelets, such as the count or proportion of large-volume platelets.

It may be understood that in this embodiment, the blood samples used to prepare the first test sample and the second test sample origin from a same patient. In a specific implementation, the blood samples used to prepare the first test sample and the second test sample may be obtained by separating a blood sample of a same patient by the blood sample supply portion 10, or the blood samples may be sampled from the same patient twice, one blood sample is used to prepare the first test sample, and the other blood sample is used to prepare the second test sample.

In the foregoing embodiments of “obtaining a first test result” and “obtaining a second test result”, there are mainly three processes involved. The first process comprises obtaining the first test result based on the optical signals of the first test sample; the second process comprises determining whether any one or a combination of reticulocytes, immature platelets and large-volume platelets in the blood sample is abnormal based on the optical signals of the first test sample; and the third process comprises preparing a second test sample and obtaining a second test result if it is determined that any one or a combination of the reticulocytes, the immature platelets and the large-volume platelets in the blood sample is abnormal. The specific contents of the three processes are separately explained below.

I. Description of obtaining the first test result based on the optical signals of the first test sample.

In some embodiments, the optical signals of the first test sample includes at least forward-scattered light signals (FSC) and fluorescence signals (FL). Similarly, the optical signals of the first test sample may further include side-scattered light signals or the like, which will not be illustrated herein one by one. Accordingly, the processor 50 controls the blood sample supply portion 10 to provide the blood sample to the mixing chamber 30, and controls the reagent supply portion 20 to provide a hemolytic agent and a first fluorescent agent to the mixing chamber 30 to prepare the first test sample in the mixing chamber 30. That is, the first test sample is obtained by treating the blood sample with the hemolytic agent and the first fluorescent agent.

It may be understood that the processor 50 obtains the first test result of the blood sample from the optical signals of the first test sample, for example, obtains a white blood cell count result and/or a white blood cell classification result of the blood sample.

The processor 50 may obtain the first test result of the blood sample in various ways, for example: the processor 50 generates a scatter diagram of the first test sample from the optical signals of the first test sample, wherein in the scatter diagram, an intensity of a forward-scattered light signal of the first test sample is taken as the abscissa and an intensity of a fluorescence signal of the first test sample is taken as the ordinate. Based on the scatter diagram, the first test result corresponding to the first test sample may be obtained.

It should be noted that the foregoing example is an example in which the forward-scattered light signals and the fluorescence signals of the first test sample are used to obtain the first test result. In other examples, the first test result may also be obtained by using side-scattered light signals and fluorescence signals of the first test sample.

Other examples in which the first test result is obtained will not be illustrated in the embodiments of the disclosure.

It should be further noted that the abscissa and the ordinate of the scatter diagram are not limited to the presentation mode of a linear coordinate axis of signal intensity mentioned above. Other parameters that can reflect the forward-scattered light signals and the fluorescence signals of particles of the first test sample may also be used as the abscissa and the ordinate of the scatter diagram, and nonlinear coordinate axes such as logarithmic coordinate axes may also be used as the abscissa and the ordinate of the scatter diagram to further highlight distribution differences of particle populations. In addition, the scatter diagram mentioned in the foregoing embodiments of the disclosure is not limited to being presented graphically, and may also be presented in the form of data, such as in the form of data of tables or lists with the same or similar resolution as that of the scatter diagram, or in any other suitable mode known in the field.

The foregoing content describes that the processor 50 controls the preparation of the first test sample, obtains the optical signals of the first test sample, and obtains the first test result including at least the white blood cell count result and/or the white blood cell classification result based on the optical signals.

II. Description of determining whether any one or a combination of reticulocytes, immature platelets and large-volume platelets in the blood sample is abnormal based on the optical signals of the first test sample.

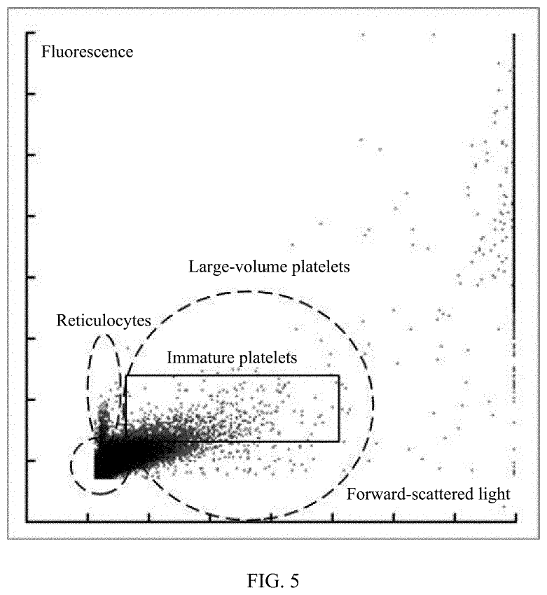

In conjunction with the foregoing content, in some embodiments, the processor 50 determines whether any one or a combination of reticulocytes, immature platelets and large-volume platelets in the blood sample is abnormal from the optical signals of the first test sample. In some specific embodiments, the processor 50 generates a scatter diagram corresponding to the first test sample from the optical signals of the first test sample, wherein the abscissa of the scatter diagram may represent the intensity of the forward-scattered light signal of the first test sample, and the ordinate thereof may represent the intensity of the fluorescence signal of the first test sample. The processor 50 obtains, on the scatter diagram of the first test sample, feature regions corresponding to the reticulocytes, the immature platelets and the large-volume platelets in the blood sample, that is, the processor obtains, on the scatter diagram of the first test sample, the feature region corresponding to the reticulocytes in the blood sample, the feature region corresponding to the immature platelets in the blood sample, and the feature region corresponding to the large-volume platelets in the blood sample. For example, FIG. 5 is a scatter diagram of a first test sample generated on the basis of forward-scattered light signals and fluorescence signals. Specifically, in the scatter diagram shown in FIG. 5, the abscissa represents the intensity of the forward-scattered light of the first test sample and the ordinate represents the intensity of the fluorescence signal of the first test sample. On the scatter diagram of the first test sample shown in FIG. 5, the feature regions corresponding to the reticulocytes, the immature platelets and the large-volume platelets in the blood sample are obtained separately.

The processor 50 determines whether any one or a combination of the reticulocytes, the immature platelets and the large-volume platelets in the blood sample is abnormal based on scatter point information in the obtained feature regions. That is, whether the reticulocytes in the blood sample are abnormal is determined based on the scatter point information in the feature region corresponding to the reticulocytes in the blood sample; whether the immature platelets in the blood sample are abnormal is determined based on the scatter point information in the feature region corresponding to the immature platelets in the blood sample; and whether the large-volume platelets in the blood sample are abnormal is determined based on the scatter point information in the feature region corresponding to the large-volume platelets in the blood sample.

In some specific embodiments, the processor 50 obtains, on the scatter diagram of the first test sample, feature regions corresponding to the reticulocytes, the immature platelets and the large-volume platelets in the blood sample.

In some specific embodiments, the processor 50 counts first scatter points in the feature region corresponding to the reticulocytes in the blood sample; the processor 50 determines that the reticulocytes in the blood sample are normal if the count of the first scatter points is within a first threshold range; and the processor 50 determines that the reticulocytes in the blood sample are abnormal if the count of the first scatter points is outside the first threshold range. It may be understood that the first threshold range may be determined on the basis of normal values of the reticulocytes. For example, if the number of scatter points in the feature region of the reticulocytes mentioned above is within the range of the normal values (that is, the count of the first scatter points is within the first threshold range), it indicates that the reticulocytes in the blood sample are normal; and if the number of scatter points in the feature region of the reticulocytes mentioned above is greater than or less than the normal value (that is, the count of the first scatter points is outside the first threshold range), it indicates that the reticulocytes in the blood sample are abnormal.

In some specific embodiments, the processor 50 counts second scatter points in the feature region corresponding to the immature platelets in the blood sample; the processor 50 determines that the immature platelets in the blood sample are normal if the count of the second scatter points is within a second threshold range; and the processor 50 determines that the immature platelets in the blood sample are abnormal if the count of the second scatter points is outside the second threshold range. It may be understood that the second threshold range is determined on the basis of normal values of the immature platelets in the blood sample. For example, if the number of scatter points in the feature region of the immature platelets mentioned above is within the range of the normal values (that is, the count of the second scatter points is within the second threshold range), it indicates that the immature platelets in the blood sample are normal; and if the number of scatter points in the feature region of the immature platelets mentioned above is greater than the normal value (that is, the count of the second scatter points is outside the second threshold range), it indicates that the immature platelets in the blood sample are abnormal.

In some specific embodiments, the processor 50 counts third scatter points in the feature region corresponding to the large-volume platelets in the blood sample; the processor 50 determines that the large-volume platelets in the blood sample are normal if the count of the third scatter points is within a third threshold range; and the processor 50 determines that the large-volume platelets in the blood sample are abnormal if the count of the third scatter points is outside the third threshold range. It may be understood that the third threshold range is determined on the basis of normal values of the large-volume platelets in the blood sample. For example, if the number of scatter points in the feature region of the large-volume platelets mentioned above is within the range of the normal values (that is, the count of the third scatter points is within the third threshold range), it indicates that the large-volume platelets in the blood sample are normal; and if the number of scatter points in the feature region of the large-volume platelets mentioned above is greater than the range of the normal values (that is, the count of the third scatter points is outside the third threshold range), it indicates that the large-volume platelets in the blood sample are abnormal.

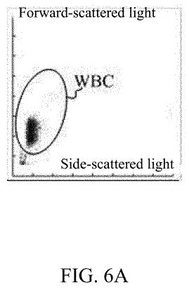

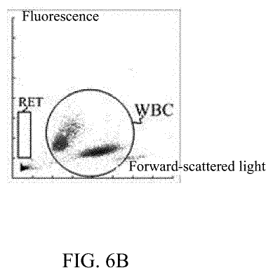

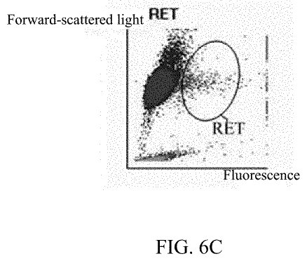

Each of FIGS. 6A and 6B shows an example in which a first test sample is prepared by using a normal blood sample (that is, a blood sample with normal reticulocytes, normal immature platelets and normal large-volume platelets).

FIG. 6A is an example of a scatter diagram of a first test sample that is generated on the basis of forward-scattered light signals and side-scattered light signals.

Specifically, in the scatter diagram (two-dimensional scatter diagram) shown in FIG. 6A, the abscissa represents an intensity of a side-scattered light signal of the first test sample and the ordinate represents an intensity of a forward-scattered light signal of the first test sample. White blood cell count can be obtained on the basis of the scatter diagram shown in FIG. 6A. A region of interest that represents scatter points of white blood cells in FIG. 6A, that is, a WBC region in FIG. 6A, has been circled.

FIG. 6B is an example of a scatter diagram of a first test sample that is generated on the basis of forward-scattered light signals and fluorescence signals. Specifically, in the scatter diagram (two-dimensional scatter diagram) shown in FIG. 6B, the abscissa represents an intensity of a forward-scattered light signal of the first test sample and the ordinate represents an intensity of a fluorescence signal of the first test sample. A white blood cell classification result can be obtained on the basis of the scatter diagram shown in FIG. 6B. Regions of interest that represent scatter points of white blood cells and reticulocytes in FIG. 6B, that is, a WBC region and a RET region in FIG. 6B, have been circled.

FIG. 6C is an example of a scatter diagram generated when a separate reticulocyte testing (for example, in the method herein, the second reaction reagent is used to prepare a second test sample and the second test sample is tested to obtain a second test result) is performed on the same blood sample (that is, the normal blood sample). Specifically, in the scatter diagram (two-dimensional scatter diagram) shown in FIG. 6C, the abscissa represents an intensity of a fluorescence signal and the ordinate represents an intensity of a forward-scattered light signal. A region of interest that represents scatter points of reticulocytes in FIG. 6C, that is, a RET region in FIG. 6C, has been circled.

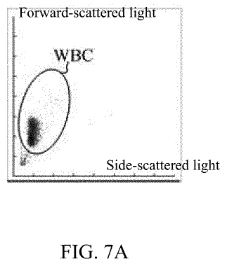

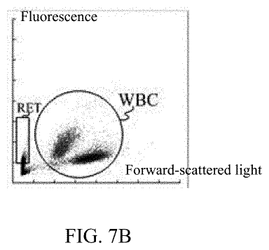

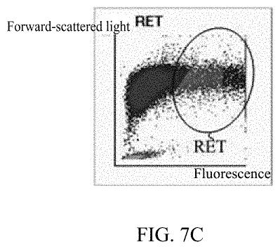

Each of FIGS. 7A and 7B shows an example in which a first test sample is prepared by using a blood sample with a high reticulocyte percentage (that is, a blood sample with abnormal reticulocytes, or an abnormal blood sample with a large number of reticulocytes).

FIG. 7A is an example of a scatter diagram of a first test sample that is generated on the basis of forward-scattered light signals and side-scattered light signals. Specifically, in the scatter diagram (two-dimensional scatter diagram) shown in FIG. 7A, the abscissa represents an intensity of a side-scattered light signal of the first test sample and the ordinate represents an intensity of a forward-scattered light signal of the first test sample. A region of interest that represents scatter points of white blood cells in FIG. 7A, that is, a WBC region in FIG. 7A, has been circled.

FIG. 7B is an example of a scatter diagram of a first test sample that is generated on the basis of forward-scattered light signals and fluorescence signals. Specifically, in the scatter diagram (two-dimensional scatter diagram) shown in FIG. 7B, the abscissa represents an intensity of a forward-scattered light signal of the first test sample and the ordinate represents an intensity of a fluorescence signal of the first test sample. A white blood cell classification result can be obtained on the basis of the scatter diagram shown in FIG. 7B. Regions of interest that represent scatter points of white blood cells and reticulocytes in FIG. 7B, that is, a WBC region and a RET region in FIG. 7B, have been circled.

FIG. 7C is an example of a scatter diagram generated when a separate reticulocyte testing (for example, in the method herein, the second reaction reagent is used to prepare a second test sample and the second test sample is tested to obtain a second test result) is performed on the same blood sample (that is, the blood sample with a high reticulocyte percentage). Specifically, in the scatter diagram (two-dimensional scatter diagram) shown in FIG. 7C, the abscissa represents an intensity of a fluorescence signal and the ordinate represents an intensity of a forward-scattered light signal. A region of interest that represents scatter points of reticulocytes in FIG. 7C, that is, a RET region in FIG. 7C, has been circled.

It is seen from the results of FIGS. 6C and 7C that the number of scatter points in the RET region in FIG. 7C is much greater than the number of scatter points in the RET region in FIG. 6C, that is, a large number of reticulocytes actually exist in the blood sample with a high reticulocyte percentage corresponding to FIG. 7C.

Therefore, for the scatter diagram of the first test sample obtained by treating the normal blood sample, the number of scatter points (or the number of particles) in the RET region is within the normal range. For the scatter diagram of the first test sample obtained by treating the blood sample with a high reticulocyte percentage, the number of scatter points in the RET region increases significantly.

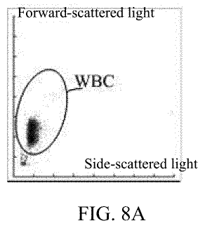

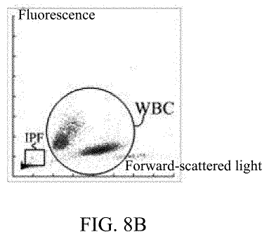

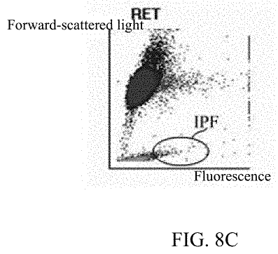

Each of FIGS. 8A and 8B shows an example in which a first test sample is prepared by using a normal blood sample (that is, a blood sample with normal reticulocytes, normal immature platelets and normal large-volume platelets).

FIG. 8A is an example of a scatter diagram of a first test sample that is generated on the basis of forward-scattered light signals and side-scattered light signals. Specifically, in the scatter diagram (two-dimensional scatter diagram) shown in FIG. 8A, the abscissa represents an intensity of a side-scattered light signal of the first test sample and the ordinate represents an intensity of a forward-scattered light signal of the first test sample. White blood cell count can be obtained on the basis of the scatter diagram shown in FIG. 8A. A region of interest that represents scatter points of white blood cells in FIG. 8A, that is, a WBC region in FIG. 8A, has been circled.

FIG. 8B is an example of a scatter diagram of a first test sample that is generated on the basis of forward-scattered light signals and fluorescence signals. Specifically, in the scatter diagram (two-dimensional scatter diagram) shown in FIG. 8B, the abscissa represents an intensity of a forward-scattered light signal of the first test sample and the ordinate represents an intensity of a fluorescence signal of the first test sample. A white blood cell classification result can be obtained on the basis of the scatter diagram shown in FIG. 8B. Regions of interest that represent scatter points of white blood cells and immature platelets in FIG. 8B, that is, a WBC region and an IPF region in FIG. 8B, have been circled.

FIG. 8C is an example of a scatter diagram generated when a separate immature platelet testing (for example, in the method herein, the second reaction reagent is used to prepare a second test sample and the second test sample is tested to obtain a second test result) is performed on the same blood sample (that is, the normal blood sample). Specifically, in the scatter diagram (two-dimensional scatter diagram) shown in FIG.

8C, the abscissa represents an intensity of a fluorescence signal and the ordinate represents an intensity of a forward-scattered light signal. A region of interest that represents scatter points of immature platelets in FIG. 8C, that is, an IPF region in FIG. 8C, has been circled.

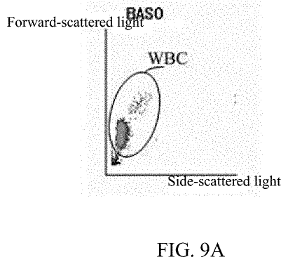

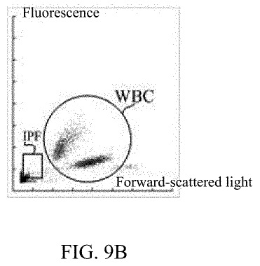

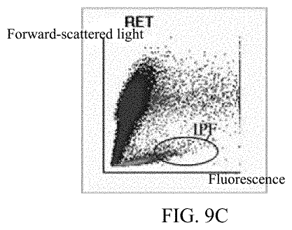

Each of FIGS. 9A and 9B shows an example in which a first test sample is prepared by using a blood sample with a high immature-platelet percentage (that is, a blood sample with abnormal immature platelets, or an abnormal blood sample with a large number of immature platelets).

FIG. 9A is an example of a scatter diagram of a first test sample that is generated on the basis of forward-scattered light signals and side-scattered light signals. Specifically, in the scatter diagram (two-dimensional scatter diagram) shown in FIG. 9A, the abscissa represents an intensity of a side-scattered light signal of the first test sample and the ordinate represents an intensity of a forward-scattered light signal of the first test sample. A region of interest that represents scatter points of white blood cells in FIG. 9A, that is, a WBC region in FIG. 9A, has been circled.

FIG. 9B is an example of a scatter diagram of a first test sample that is generated on the basis of forward-scattered light signals and fluorescence signals. Specifically, in the scatter diagram (two-dimensional scatter diagram) shown in FIG. 9B, the abscissa represents an intensity of a forward-scattered light signal of the first test sample and the ordinate represents an intensity of a fluorescence signal of the first test sample. A white blood cell classification result can be obtained on the basis of the scatter diagram shown in FIG. 9B. Regions of interest that represent scatter points of white blood cells and immature platelets in FIG. 9B, that is, a WBC region and an IPF region in FIG. 9B, have been circled.

FIG. 9C is an example of a scatter diagram generated when a separate immature platelet testing (for example, in the method herein, the second reaction reagent is used to prepare a second test sample and the second test sample is tested to obtain a second test result) is performed on the same blood sample (that is, the blood sample with a high immature platelet percentage). Specifically, in the scatter diagram (two-dimensional scatter diagram) shown in FIG. 9C, the abscissa represents an intensity of a fluorescence signal and the ordinate represents an intensity of a forward-scattered light signal. A region of interest that represents scatter points of immature platelets in FIG. 9C, that is, an IPF region in FIG. 9C, has been circled.

It is seen from the results of FIGS. 8C and 9C that the number of scatter points in the IPF region in FIG. 9C is much greater than the number of scatter points in the IPF region in FIG. 8C, that is, a large number of immature platelets actually exist in the blood sample with a high immature platelet percentage corresponding to FIG. 9C.

Therefore, for the scatter diagram of the first test sample obtained by treating the normal blood sample, the number of scatter points (or the number of particles) in the IPF region is within the normal range. For the scatter diagram of the first test sample obtained by treating the blood sample with a high immature platelet percentage, the number of scatter points in the IPF region increases significantly.

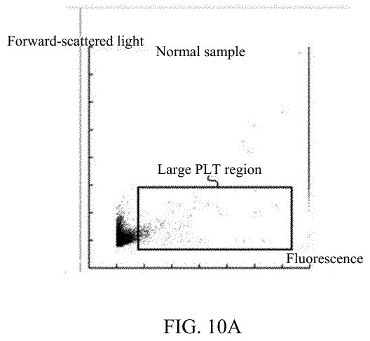

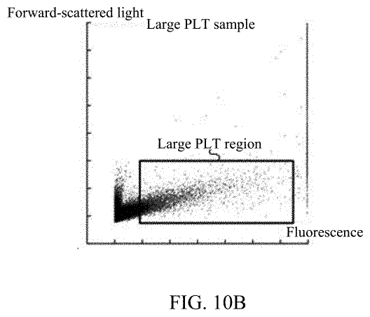

FIG. 10A is an example of a scatter diagram generated when a large volume platelet testing (for example, in the method herein, the second reaction reagent is used to prepare a second test sample and the second test sample is tested to obtain a second test result) is performed on a normal blood sample (that is, a blood sample with normal reticulocytes, immature platelets and large-volume platelets). Specifically, in the scatter diagram (two-dimensional scatter diagram) shown in FIG. 10A, the abscissa represents an intensity of a fluorescence signal and the ordinate represents an intensity of a forward-scattered light signal. A region of interest that represents scatter points of large-volume platelets in FIG. 10A, that is, a large PLT region in FIG. 10A, has been circled.

FIG. 10B is an example of a scatter diagram generated when a large volume platelet testing (for example, in the method herein, the second reaction reagent is used to prepare a second test sample and the second test sample is tested to obtain a second test result) is performed on a blood sample with a high large volume platelet percentage (that is, a blood sample with abnormal large-volume platelets, or an abnormal blood sample with a large number of large-volume platelets). Specifically, in the scatter diagram (two-dimensional scatter diagram) shown in FIG. 10B, the abscissa represents an intensity of a fluorescence signal and the ordinate represents an intensity of a forward-scattered light signal. A region of interest that represents scatter points of large-volume platelets in FIG. 10B, that is, a large PLT region in FIG. 10B, has been circled. It is seen from the results of FIGS. 10A and 10B that the number of scatter points in the large PLT region in FIG. 10B is much greater than the number of scatter points in the large PLT region in FIG. 10A, that is, a large number of large-volume platelets actually exist in the blood sample with a high large-volume platelet percentage corresponding to FIG. 10B.

Therefore, for the scatter diagram obtained by treating the normal blood sample, the number of scatter points (or the number of particles) in the large PLT region is within the normal range. For the scatter diagram obtained by treating the blood sample with a high large-volume-platelet percentage, the number of scatter points in the large PLT region increases significantly.

The foregoing content describes the process and principle of determining whether any one or a combination of reticulocytes, immature platelets and large-volume platelets in the blood sample is abnormal based on the optical signals of the first test sample, and verifies the process and principle by separately testing any one or a combination of reticulocytes, immature platelets and large-volume platelets in the blood sample. Therefore, as described above, in some embodiments, if it is determined that any one or a combination of the reticulocytes, the immature platelets and the large-volume platelets in the blood sample is abnormal, the processor 50 controls preparation, obtains optical signals of the second test sample, and obtains a second test result of the blood sample based on the optical signals of the second test sample, wherein the second test result includes any one or a combination of a reticulocyte parameter, an immature platelet parameter and a large-volume platelet parameter of the second test sample, which will be described in detail below.

III. Description of preparing a second test sample and obtaining a second test result if it is determined that any one or a combination of the reticulocytes, the immature platelets and the large-volume platelets in the blood sample is abnormal.

if it is determined that any one or a combination of the reticulocytes, the immature platelets and the large-volume platelets in the blood sample is abnormal, the processor 50 controls the blood sample supply portion 10 to provide the blood sample to the mixing chamber 30, and controls the reagent supply portion 20 to provide a second reaction reagent to the mixing chamber 30 to prepare a second test sample in the mixing chamber 30, wherein the second reaction reagent contains no hemolytic agent. The processor 50 controls the optical detection portion 69 to obtain optical signals of the second test sample, and obtains a second test result of the blood sample from the optical signals of the second test sample. It may be understood that the first test result has been obtained by using the first test sample before the second test result is obtained, and the first test result obtained before is also output when the second test result is obtained, wherein the second test result includes any one or a combination of a reticulocyte parameter, an immature platelet parameter and a large-volume platelet parameter of the second test sample. It should be noted that, in the embodiment described in this paragraph, the blood sample for preparing the second test sample and the blood sample for preparing the first test sample both origin from a same patient.

In some embodiments, the optical signals of the second test sample includes forward-scattered light signals (FSC) and fluorescence signals (FL). In this case, the processor 50 controls the blood sample supply portion 10 to provide the blood sample to the mixing chamber, and controls the reagent supply portion 20 to provide a diluent and a second fluorescent agent to the mixing chamber 30 to prepare the second test sample in the mixing chamber 30, that is, the second test sample is obtained by treating the blood sample with the diluent and the second fluorescent agent.

In some embodiments, the processor 50 obtains the second test result of the blood sample from the optical signals of the second test sample, such as one or a combination of a reticulocyte counting result, an immature platelet count result and a large-volume platelet count result (the results that need to be obtained are determined according to actual requirements), and there may be many ways to obtain the second test result.

In some specific embodiments, the processor 50 generates a scatter diagram corresponding to the second test sample from the optical signals of the second test sample, and obtains the second test result of the blood sample according to the scatter diagram. For example, FIGS. 7C, 9C and 10B are some examples. Each of FIGS. 7C, 9C and 10B isa scatter diagram of a second test sample generated on the basis of forward-scattered light signals and fluorescence signals. Specifically, in the scatter diagram, the abscissa represents an intensity of a fluorescence signal of the second test sample and the ordinate represents an intensity of a forward-scattered light signal of the second test sample.

Based on the scatter diagram corresponding to FIG. 7C, a reticulocyte parameter (such as a counting result) of the second test sample may be obtained; based on the scatter diagram corresponding to FIG. 9C, an immature platelet parameter of the second test sample may be obtained; and based on the scatter diagram corresponding to FIG. 10B, a large-volume platelet parameter of the second test sample may be obtained. The above descriptions are examples in which the forward-scattered light signals and the fluorescence signals of the second test sample are used to obtain the second test result.

The above content is the description of preparing and measuring the second test sample if it is determined that any one or a combination of the reticulocytes, the immature platelets and the large-volume platelets in the blood sample is abnormal. In some embodiments, the processor 50 further generates a corresponding alarm prompt if it is determined that any one or a combination of the reticulocytes, the immature platelets and the large-volume platelets in the blood sample is abnormal. As an example, when the reticulocytes in the blood sample are abnormal, the processor 50 sends an alarm prompt for indicating abnormal reticulocytes. As another example, when the immature platelets in the blood sample are abnormal, the processor 50 sends an alarm prompt for indicating abnormal immature platelets. As yet another example, when the large-volume platelets in the blood sample are abnormal, the processor 50 sends an alarm prompt for indicating abnormal large-volume platelets. It should be noted that a specific alarm prompt mode may be set by a technician according to actual needs, for example, the blood sample may be specially marked in a test report or a displayed test image, or a user may be informed in the form of sound, flash or the like.

Application scenarios of the disclosure will be described below in conjunction with some physical devices.

A general blood cell analyzer or blood analysis apparatus may include one or more test channels. White blood cell counting by using a laser scattering method or an optical method may act as an independent test channel, which may be referred to as a white blood cell hemolysis channel or a WBC counting channel. White blood cell classification by using the laser scattering method or the optical method may act as an independent test channel, for example, a DIFF channel in a related instrument from Mindray is a test channel for white blood cell classification. Red blood cell counting by using an impedance method may act as an independent test channel, which may be referred to as a red blood cell impedance channel. Platelet counting by using the impedance method may act as an independent test channel, which may be referred to as a platelet impedance channel. In some examples, the red blood cell impedance channel and the platelet impedance channel may be the same impedance channel. Platelet measurement by using the optical method may also act as an independent test channel, which may be denoted as R, and this test channel may be used to test reticulocytes, immature platelets, and large-volume platelets. Measurement or counting of nucleated red blood cells may also act as an independent test channel, which may be denoted as N.

To quickly select a combination of one or more test channels, some measurement modes are defined in general blood cell analyzers, and these measurement modes are the combination of the foregoing test channels. For example, a CBC measurement mode generally includes white blood cell counting, red blood cell counting by using the impedance method, and platelet counting by using the impedance method; a CD mode generally includes white blood cell counting, white blood cell classification, red blood cell counting by using the impedance method, and platelet counting by using the impedance method; an R measurement mode is to test platelets, reticulocytes, and immature platelets; and an N measurement mode is to count nucleated red blood cells.

The CBC measurement mode may be combined with the R measurement mode to form a new measurement mode (which may be referred to as a CR measurement mode), and the CD measurement mode may be combined with the R measurement mode to form a new measurement mode (which may be referred to as a CDR measurement mode). Similarly, the CBC measurement mode and the CD measurement mode may also be separately combined with the N measurement mode to form new measurement modes. Further, the CBC measurement mode and the CD measurement mode may be combined with the N measurement mode and the R measurement mode to form new measurement modes.

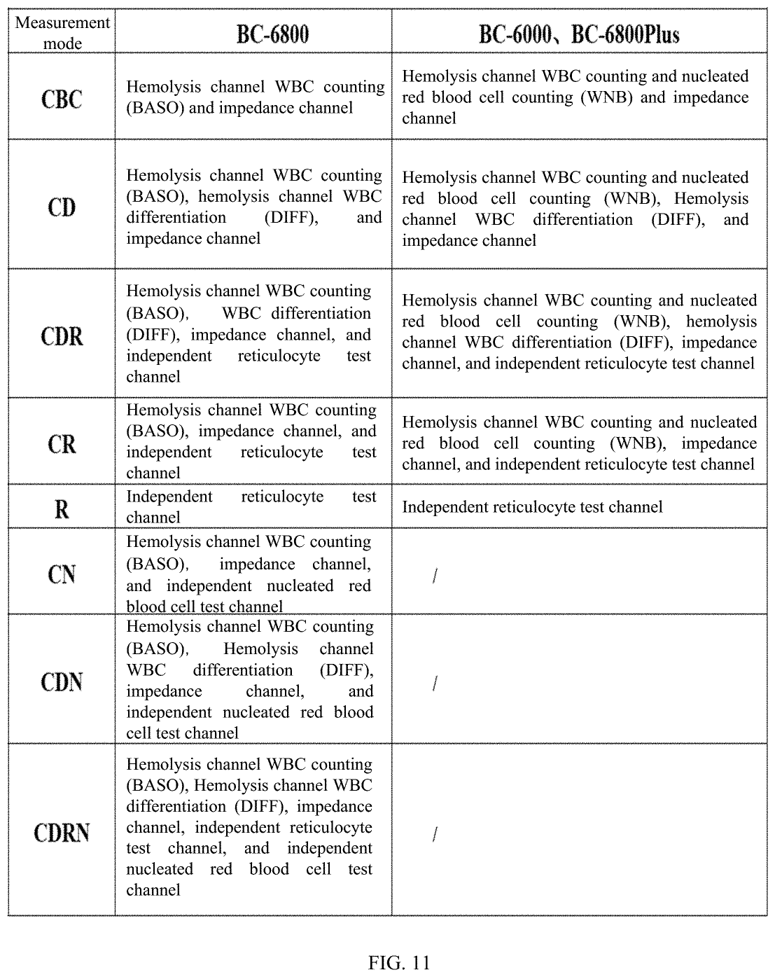

The CDR measurement mode of some instruments, such as BC-6000 and BC-6800Plus blood cell analyzers produced by Shenzhen Mindray Bio-medical Electronics Co., Ltd. includes a measurement function of a CDRN mode . FIG. 11 illustrates some measurement modes of a BC-6800 blood cell analyzer, a BC-6000 blood cell analyzer and a BC-6800Plus blood cell analyzer produced by Shenzhen Mindray Bio-medical Electronics Co., Ltd.

The CBC measurement mode of the BC-6800 blood cell analyzer can perform white blood cell counting, red blood cell counting, and platelet counting; the CD measurement mode can perform white blood cell counting and classification, red blood cell counting, and platelet counting; the CDR measurement mode can perform white blood cell counting and classification, red blood cell counting, platelet counting, and reticulocyte counting; the CR measurement mode can perform white blood cell counting, red blood cell counting, platelet counting, and reticulocyte counting; the R measurement mode can complete the measurement of reticulocytes, immature platelets, and large-volume platelets; the CN measurement mode can perform white blood cell counting, red blood cell counting, platelet counting, and nucleated red blood cell counting; the CDN measurement mode can perform white blood cell counting and classification, red blood cell counting, platelet counting, and nucleated red blood cell counting; and the CDRN measurement mode can perform white blood cell counting and classification, red blood cell counting, platelet counting, reticulocyte counting, and nucleated red blood cell counting.

With respect to FIG. 11 and the foregoing content, it may be seen that the measurement modes of the BC-6800 blood cell analyzer other than some measurement modes (such as the R measurement mode) including only one test channel each generally have a WBC count channel or a DIFF channel. Therefore, when the disclosure is applied to the BC-6800 blood cell analyzer, it may be determined whether any one or a combination of the reticulocytes, the immature platelets and the large-volume platelets in the blood sample is abnormal from optical signals of the test sample for a test on white blood cell counting and/or classification. If any one or a combination of the reticulocytes, the immature platelets and the large-volume platelets in the blood sample is abnormal, a separate channel (namely an R channel, also referred to as independent reticulocyte test channel) for the reticulocytes, the immature platelets and the large-volume platelets is opened to count any one or a combination of the reticulocytes, the immature platelets and the large-volume platelets. That is, the R channel may be in a normally closed state, and only if it is determined that any one or a combination of the reticulocytes, the immature platelets and the large-volume platelets in the blood sample is abnormal from the optical signals of the test sample for a test on white blood cell counting and/or classification during conventional measurement, the R channel is opened to more accurately count any one or a combination of the reticulocytes, the immature platelets and the large-volume platelets. Therefore, the BC-6800 blood cell analyzer may keep the WBC count channel or the DIFF channel normally open, and then determine whether to open the R channel based on the optical signals obtained from the normally open test channel.

The white blood cell hemolysis channel (or WBC count channel) and the nucleated red blood cell count channel (N channel) of the BC-6000 and BC-6800Plus blood analysis analyzers is a same channel, which may be referred to as a WNB channel, and white blood cell counting and nucleated red blood cell counting may be completed by using the WNB channel. Specifically, the CBC measurement mode of the BC-600 and

BC-6800Plus blood cell analyzers may perform white blood cell counting, red blood cell counting, platelet counting, and nucleated red blood cell counting; the CD measurement mode may perform white blood cell counting and classification, red blood cell counting, platelet counting, and nucleated red blood cell counting; and the CDR measurement mode may perform white blood cell counting and classification, red blood cell counting, platelet count, nucleated red blood cell counting, and reticulocyte counting; and the CR measurement mode may perform white blood cell counting, red blood cell counting, platelet counting, nucleated red blood cell counting, and reticulocyte counting.

With respect to FIG. 11 and the foregoing content, it may be seen that the measurement modes of the BC-6000 and BC-6800Plus blood cell analyzers other than some measurement modes (such as the R measurement mode) including only one test channel each generally have a WBC count channel or a DIFF channel. Therefore, when the disclosure is applied to the BC-6000 or BC-6800Plus blood cell analyzers, whether to open the R channel may be determined from the optical signals of the test sample for a test on white blood cell counting and/or classification.

The above describes that whether to open the R channel may be determined on the basis of the optical signals obtained in the opened test channel, when the separate channel (R channel) for reticulocyte counting, immature platelet counting and large-volume platelet counting is not opened and the channel for white blood cell counting and/or classification is opened. It may be understood that in the disclosure, there are many application scenarios in which it is determined whether to open the R channel based on optical signals obtained in the channel for white blood cell counting and/or classification, because the channel for white blood cell counting and/or classification generally needs to be opened when a blood sample is tested, that is, relevant test items for white blood cell counting and/or classification generally need to be performed when a blood sample is tested.

In some other embodiments, whether the R channel is opened may be determined first. If the R channel is opened, any one or a combination of a reticulocyte parameter, an immature platelet parameter and a large-volume platelet parameter is directly output, and if the R channel is not opened, whether to open the R channel is determined based on the optical signals obtained in the channel for white blood cell counting and/or classification. Details are described below.

In some embodiments, the blood analysis apparatus includes a first measurement mode in which no R channel is included and a second measurement mode in which at least the R channel is included; and the processor 50 determines whether the second measurement mode is enabled (that is, whether the R channel is opened). Taking the BC-6800 blood analyzer as an example, if the CDR measurement mode, the CR measurement mode, the R measurement mode or the CDRN measurement mode is enabled, it indicates that the second measurement mode is enabled (that is, the R channel is in an open state). Similarly, taking the BC-6800 blood analyzer as an example, if the CBC measurement mode, the CD measurement mode, the CN measurement mode or the CDN measurement mode is enabled, it indicates that the second measurement mode is not enabled (that is, the R channel is not opened).

If the second measurement mode is enabled, the processor 50 controls the blood sample supply portion 10 to provide the blood sample to the mixing chamber 30, and controls the reagent supply portion 20 to provide a second reaction reagent to the mixing chamber 30 to prepare a second test sample in the mixing chamber 30, wherein the second reaction reagent contains no hemolytic agent; and the processor 50 controls the optical detection portion 69 to obtain optical signals of the second test sample, and obtains a second test result of the blood sample from the optical signal of the second test sample, wherein the second test result includes any one or a combination of a reticulocyte parameter, an immature platelet parameter and a large-volume platelet parameter of the second test sample. How to prepare the second test sample, how to obtain the optical signals and its type of the second test sample, and how to obtain the second test result from the optical signals have been described in detail above, and are not described again.

If the second measurement mode is not enabled, in the first measurement mode (that is, in the measurement mode with no R channel opened), the processor 50 controls the blood sample supply portion 10 to provide the blood sample to the mixing chamber 30, and controls the reagent supply portion 20 to provide a first reaction reagent to the mixing chamber 30 to prepare a first test sample in the mixing chamber, wherein the first reaction reagent contains a hemolytic agent. The processor 50 controls the optical detection portion 69 to obtain optical signals of the first test sample, and determines a first test result of the blood sample from the optical signals of the first test sample, wherein the first test result includes a white blood cell count result and/or a white blood cell classification result; wherein the first test sample is prepared from the blood sample provided by the blood supply portion 10 and at least the first reaction reagent provided by the reagent supply portion 20, which are received by the mixing chamber 30. The processor 50 determines whether to enable the second measurement mode by determining whether any one or a combination of the reticulocytes, the immature platelets and the large-volume platelets in the blood sample is abnormal from the optical signals of the first test sample (that is, whether the R channel should be opened is determined). If it is determined that any one or a combination of the reticulocytes, the immature platelets and the large-volume platelets in the blood sample is abnormal, it is determined that the second measurement mode should be enabled, the second measurement mode is enabled, a second test sample is prepared, optical signals of the second test sample are obtained, a second test result of the blood sample is obtained from the optical signals of the second test sample, and the first test result is output; and if it is determined that the reticulocytes, the immature platelets and the large-volume platelets in the blood sample are all normal, it is determined that the second measurement mode should not be enabled and the first test result is output. The specific content described in this paragraph has been described in detail above, and will not be described again. It may be understood that the blood samples for preparing the first test sample and the second test sample origin from a same patient.

The above content involves in determining whether the R channel is opened. If the R channel is opened, any one or a combination of the reticulocyte parameter, the immature platelet parameter and the large-volume platelet parameter will be directly output. If the R channel is not opened, whether to open the R channel is determined based on the optical signals obtained in the channel for white blood cell counting and/or classification.

The above is the description of the blood analysis apparatus. A blood analysis method is further disclosed in some embodiments of the disclosure.

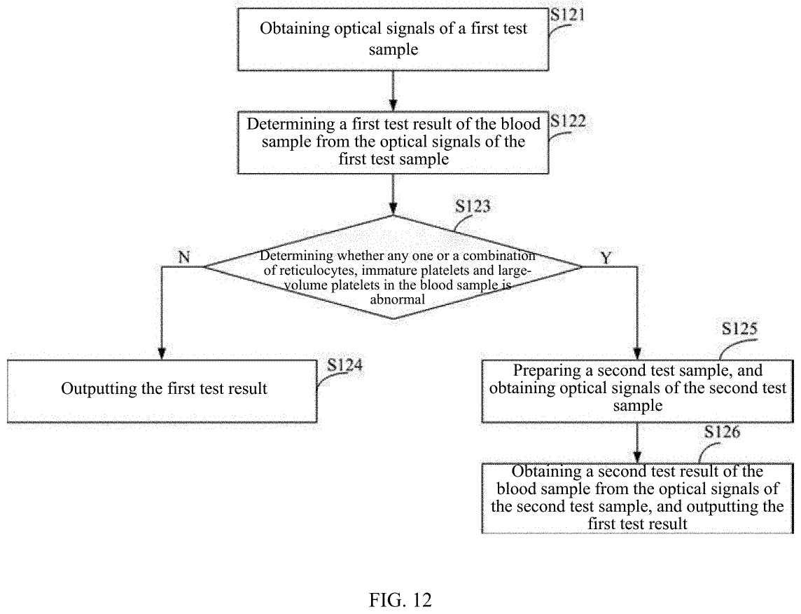

Referring to FIG. 12, the blood analysis method in some embodiments includes the following steps.

Step S121: obtaining optical signals of a first test sample, wherein the first test sample is obtained by treating a blood sample with at least a first reaction reagent, and the first reaction reagent contains a hemolytic agent.

In an embodiment, the first test sample is obtained by treating the blood sample with the hemolytic agent and a first fluorescent agent, and the optical signals of the first test sample includes forward-scattered light signals and fluorescence signals.

Step S122: determining a first test result of the blood sample from the optical signals of the first test sample, wherein the first test result comprises a white blood cell count result and/or a white blood cell classification result.

Step S123: determining whether any one or a combination of reticulocytes, immature platelets and large-volume platelets in the blood sample is abnormal from the optical signals of the first test sample.

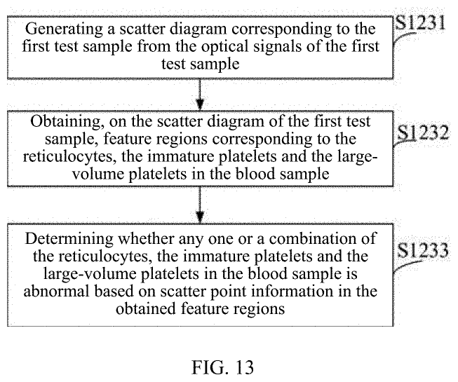

Referring to FIG. 13, in some embodiments, step S123 of determining whether any one or a combination of reticulocytes, immature platelets and large-volume platelets in the blood sample is abnormal from the optical signals of the first test sample includes the following steps:

Step S1231: generating a scatter diagram corresponding to the first test sample from the optical signals of the first test sample.

Step S1232: obtaining, on the scatter diagram of the first test sample, feature regions corresponding to the reticulocytes, the immature platelets and the large-volume platelets in the blood sample.

Step S1233: determining whether any one or a combination of the reticulocytes, the immature platelets and the large-volume platelets in the blood sample is abnormal based on scatter point information in the obtained feature regions.

During specifically implementing step S1233, first scatter points in the feature region corresponding to the reticulocytes in the blood sample are counted; and it is determined that the reticulocytes in the blood sample are normal if the count of the first scatter points is within a first threshold range; and it is determined that the reticulocytes in the blood sample are abnormal if the count of the first scatter points is outside the first threshold range.

Second scatter points in the feature region corresponding to the immature platelets in the blood sample are counted; and it is determined that the immature platelets in the blood sample are normal if the count of the second scatter points is within a second threshold range; and it is determined that the immature platelets in the blood sample are abnormal if the count of the second scatter points is outside the second threshold range.

Third scatter points in the feature region corresponding to the large-volume platelets in the blood sample are counted; and it is determined that the large-volume platelets in the blood sample are normal if the count of the third scatter points is within a third threshold range; and it is determined that the large-volume platelets in the blood sample are abnormal if the count of the third scatter points is outside the third threshold range.

Step S124: outputting the first test result if it is determined that the reticulocytes, the immature platelets and the large-volume platelets in the blood sample are all normal.

Step S125: if it is determined that any one or a combination of the reticulocytes, the immature platelets and the large-volume platelets in the blood sample is abnormal, preparing a second test sample, and obtaining optical signals of the second test sample, wherein the second test sample is obtained by treating the blood sample with at least a second reaction reagent, and the second reaction reagent contains no hemolytic agent.

In some embodiments, the second test sample is obtained by treating the blood sample with a diluent and a second fluorescent agent, and the optical signals of the second test sample includes forward-scattered light signals and fluorescence signals.

In some embodiments, a corresponding alarm prompt is further generated if it is determined that any one or a combination of the reticulocytes, the immature platelets and the large-volume platelets in the blood sample is abnormal.

Step S126: obtaining a second test result of the blood sample from the optical signal of the second test sample, and outputting the first test result I, wherein the second test result includes any one or a combination of a reticulocyte parameter, an immature platelet parameter and a large-volume platelet parameter of the second test sample.

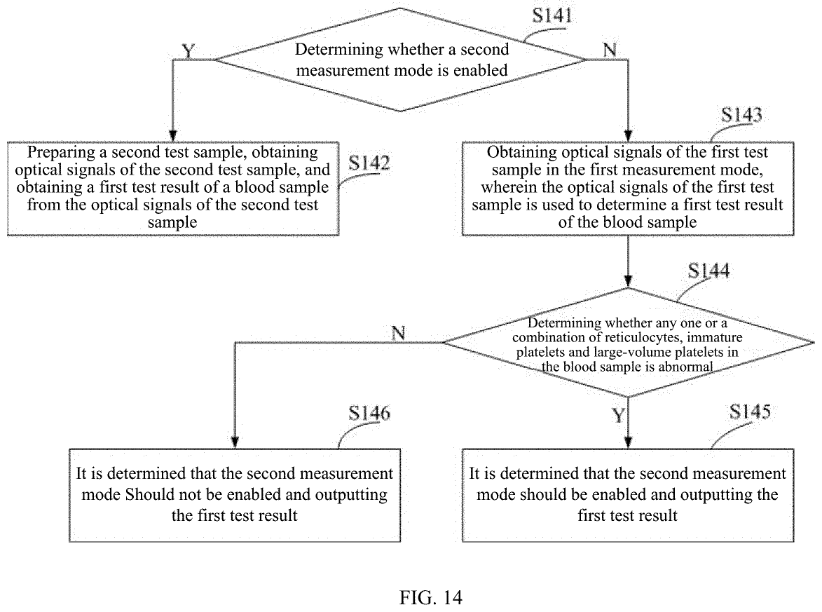

Referring to FIG. 14, a blood analysis method in some embodiments includes the following steps.

Step S141: determining whether a second measurement mode is enabled, where a blood analysis apparatus includes a first measurement mode and a second measurement mode.

Step S142: if the second measurement mode is enabled, preparing a second test sample, obtaining optical signals of the second test sample, and obtaining a second test result of a blood sample from the optical signals of the second test sample, wherein the second test result includes any one or a combination of a reticulocyte parameter, an immature-platelet parameter and a large-volume-platelet parameter of the second test sample, the second test sample is obtained by treating the blood sample with at least a second reaction reagent, and the second reaction reagent contains no hemolytic agent.

Step S143: if the second measurement mode is not enabled, obtaining optical signals of a first test sample in the first measurement mode, where the optical signals of the first test sample is used to determine a first test result of the blood sample, the first test result includes a white blood cell count result and/or a white blood cell classification result, the first test sample is obtained by treating the blood sample with at least a first reaction reagent, and the first reaction reagent contains a hemolytic agent.

Step S144: determining whether to enable the second measurement mode by determining whether any one or a combination of reticulocytes, immature platelets and large-volume platelets in the blood sample is abnormal from the optical signals of the first test sample.

Step S145: if it is determined that any one or a combination of the reticulocytes, the immature platelets and the large-volume platelets in the blood sample is abnormal, it is determined that the second measurement mode should be enabled and the first test result is output.

Step S146: if it is determined that the reticulocytes, the immature platelets and the large-volume platelets in the blood sample are all normal, it is determined that the second measurement mode should not be enabled and the first test result is output.

It should be noted that the principle of carrying out each step in FIG. 14 has been described in detail in the blood analysis apparatus above, and will not be described herein again.

It can be seen from the foregoing content that after the application of the disclosure, an application scenario comes as follows: in a conventional measurement mode (for example, a measurement mode in which the R channel for white blood cell counting or white blood cell classification or the like is not opened), it may be automatically determined whether the R channel needs to be opened for testing; that is, for an ordinary patient, there is no need to open the independent reticulocyte test channel (namely, the R channel), so the ordinary patient does not need to consume an extra reagent, which lowers test costs and improves test efficiency, and also prevents misdiagnosis and missed diagnosis of diseases related to reticulocytes, immature platelets and large-volume platelets.

The description has been made with reference to various exemplary embodiments herein. However, persons skilled in the art would have appreciated that changes and modifications could have been made to the exemplary embodiments without departing from the scope herein. For example, various operation steps and assemblies for executing operation steps may be implemented in different ways according to a specific application or considering any number of cost functions associated with the operation of the system (for example, one or more steps may be deleted, modified or incorporated into other steps).

In the foregoing embodiments, the disclosure may be implemented in whole or in part by software, hardware, firmware, or any combination thereof. In addition, as understood by persons skilled in the art, the principles herein may be reflected in a computer program product on a computer-readable storage medium that is pre-installed with computer-readable program code. Any tangible, non-transitory computer-readable storage medium can be used, including magnetic storage devices (hard disks, floppy disks, etc.), optical storage devices (CD-ROM, DVD, Blu Ray disks, etc.), flash memories, and/or the like. These computer program instructions can be loaded onto a general-purpose computer, a dedicated computer, or other programmable data processing apparatus to form a machine, such that these instructions executed on a computer or other programmable data processing apparatus can generate an apparatus that implements a specified function. These computer program instructions can also be stored in a computer-readable memory that can instruct a computer or other programmable data processing apparatus to operate in a specific manner, such that the instructions stored in the computer-readable memory can form a manufactured product, including an implementation apparatus that implements a specified function. The computer program instructions may also be loaded onto a computer or other programmable data processing apparatus, such that a series of operating steps are executed on the computer or other programmable device to produce a computer-implemented process, such that the instructions executed on the computer or other programmable device can provide steps for implementing a specified function.

Although the principles herein have been shown in various embodiments, many modifications of structures, arrangements, ratios, elements, materials, and components that are particularly suitable for specific environments and operating requirements can be made without departing from the principles and scope of the disclosure. The foregoing modifications and other changes or amendments will fall within the scope herein.

The above specific description has been described with reference to various embodiments. However, persons skilled in the art would have appreciated that various modifications and changes could have been made without departing from the scope of the disclosure. Therefore, consideration of the disclosure will be in an illustrative rather than a restrictive sense, and all such modifications will be included within the scope thereof. Likewise, the advantages of various embodiments, other advantages, and the solutions to problems have been described above. However, the benefits, advantages, solutions to problems, and any elements that can produce these, or solutions that make them more explicit, should not be interpreted as critical, necessary, or essential. The term “comprise”, “include”, and any other variants thereof used herein are non-exclusive, such that the process, method, document, or device that includes a list of elements includes not only these elements, but also other elements that are not explicitly listed or do not belong to the process, method, system, document, or device. Furthermore, the term “coupling” and any other variations thereof used herein refer to physical connection, electrical connection, magnetic connection, optical connection, communication connection, functional connection, and/or any other connection.

Persons skilled in the art will recognize that many changes may be made to the details of the above-described embodiments without departing from the basic principles of the disclosure. Therefore, the scope of the disclosure should be determined by only the claims.

1-15. (canceled)

16. A blood analysis apparatus, comprising:
a blood sample supply portion configured to provide a blood sample;
a reagent supply portion configured to provide a reaction reagent;
at least one mixing chamber configured to receive the blood sample provided by the blood sample supply portion and the reaction reagent provided by the reagent supply portion to prepare a test sample;
a measuring portion comprising an optical detection portion, wherein the optical detection portion comprises a flow chamber, a light source, and an optical detector;
wherein the flow chamber communicates with the mixing chamber to allow cells of the test sample to pass therethrough one by one, the light source is configured to irradiate the cells passing through the flow chamber, and the optical detector is configured to obtain optical signals of the cells passing through the flow chamber; and
the blood analysis apparatus further comprises a first measurement mode and a second measurement mode, and the blood analysis apparatus further comprises a processor, wherein
the processor determines whether the second measurement mode is enabled, if the second measurement mode is enabled, the processor controls the blood sample supply portion to provide the blood sample to the mixing chamber, and controls the reagent supply portion to provide a second reaction reagent to the mixing chamber to prepare a second test sample in the mixing chamber, wherein the second reaction reagent contains no hemolytic agent; the processor further controls the optical detection portion to obtain optical signals of the second test sample and obtains a second test result of the blood sample from the optical signals of the second test sample, wherein the second test result comprises any one or a combination of a reticulocyte parameter, an immature platelet parameter and a large-volume platelet parameter of the second test sample; and
if the second measurement mode is not enabled, in the first measurement mode, the processor controls the blood sample supply portion to provide the blood sample to the mixing chamber, and controls the reagent supply portion to provide a first reaction reagent to the mixing chamber to prepare a first test sample in the mixing chamber, wherein the first reaction reagent contains a hemolytic agent; the processor further controls the optical detection portion to obtain optical signals of the first test sample, and determines a first test result of the blood sample from the optical signals of the first test sample, wherein the first test result of the blood sample comprises a white blood cell count result and/or a white blood cell classification result; wherein the first test sample is prepared from the blood sample provided by the blood supply portion and at least the first reaction reagent provided by the reagent supply portion, which are received by the mixing chamber; the processor further determines whether to enable the second measurement mode by determining whether any one or a combination of reticulocytes, immature platelets and large-volume platelets in the blood sample is abnormal from the optical signals of the first test sample; and if it is determined that any one or a combination of the reticulocytes, the immature platelets and the large-volume platelets in the blood sample is abnormal, it is determined that the second measurement mode should be enabled and the first test result is output; if it is determined that the reticulocytes, the immature platelets and the large-volume platelets in the blood sample are all normal, it is determined that the second measurement mode should not be enabled and the first test result is output.

17. The apparatus of claim 16, wherein the optical signals of the first test sample comprise forward-scattered light signals and fluorescence signals, wherein the processor further controls the blood sample supply portion to provide the blood sample to the mixing chamber and controls the reagent supply portion to provide the hemolytic agent and a first fluorescent agent to the mixing chamber to prepare the first test sample in the mixing chamber.

18. The apparatus of claim 16, wherein the processor determining whether any one or a combination of the reticulocytes, the immature platelets and the large-volume platelets in the blood sample is abnormal from the optical signals of the first test sample comprises:
the processor generating a scatter diagram corresponding to the first test sample from the optical signals of the first test sample;
the processor obtaining, on the scatter diagram of the first test sample, feature regions corresponding to the reticulocytes, the immature platelets and the large-volume platelets in the blood sample; and
the processor determining whether any one or a combination of the reticulocytes, the immature platelets and the large-volume platelets in the blood sample is abnormal based on scatter point information in the obtained feature regions.

19. The apparatus of claim 18, wherein the processor determining whether any one or a combination of the reticulocytes, the immature platelets and the large-volume platelets in the blood sample is abnormal based on scatter point information in the obtained feature regions comprises:
the processor counting first scatter points in the feature region corresponding to the reticulocytes in the blood sample;
the processor determining that the reticulocytes in the blood sample are normal if the count of the first scatter points is within a first threshold range;
the processor determining that the reticulocytes in the blood sample are abnormal if the count of the first scatter points is outside the first threshold range;
the processor counting second scatter points in the feature region corresponding to the immature platelets in the blood sample;
the processor determining that the immature platelets in the blood sample are normal if the count of the second scatter points is within a second threshold range;
the processor determining that the immature platelets in the blood sample are abnormal if the count of the second scatter points is outside the second threshold range;
the processor counting third scatter points in the feature region corresponding to the large-volume platelets in the blood sample; and
the processor determining that the large-volume platelets in the blood sample are normal if the count of the third scatter points is within a third threshold range;
the processor determining that the large-volume platelets in the blood sample are abnormal if the count of the third scatter points is outside the third threshold range.

20. The apparatus of claim 16, wherein the processor further generates a corresponding alarm prompt if it is determined that any one or a combination of the reticulocytes, the immature platelets and the large-volume platelets in the blood sample is abnormal.

21. The apparatus of claim 16, wherein the optical signals of the second test sample comprises forward-scattered light signals and fluorescence signals, wherein the processor further controls the blood sample supply portion to provide the blood sample to the mixing chamber and controls the reagent supply portion to provide a diluent and a second fluorescent agent to the mixing chamber to prepare the second test sample in the mixing chamber.

22. A blood analysis apparatus, comprising:
a blood sample supply portion configured to provide a blood sample;
a reagent supply portion configured to provide a reaction reagent;
at least one mixing chamber configured to receive the blood sample provided by the blood sample supply portion and the reaction reagent provided by the reagent supply portion to prepare a test sample;
a measuring portion comprising an optical detection portion, wherein the optical detection portion comprises a flow chamber, a light source, and an optical detector;
wherein the flow chamber communicates with the mixing chamber to allow cells of the test sample to pass therethrough one by one, the light source is configured to irradiate the cells passing through the flow chamber, and the optical detector is configured to obtain optical signals of the cells passing through the flow chamber; and
a processor, wherein
the processor controls the blood sample supply portion to provide the blood sample to the mixing chamber, and controls the reagent supply portion to provide a first reaction reagent to the mixing chamber to prepare a first test sample in the mixing chamber, wherein the first reaction reagent contains a hemolytic agent; the processor further controls the optical detection portion to obtain optical signals of the first test sample, and determines a first test result of the blood sample from the optical signals of the first test sample, wherein the first test result of the blood sample comprises a white blood cell count result and/or a white blood cell classification result;
the processor further determines whether any one or a combination of reticulocytes, immature platelets and large-volume platelets in the blood sample is abnormal from the optical signals of the first test sample;
the processor further outputs the first test result if it is determined that the reticulocytes, the immature platelets and the large-volume platelets in the blood sample are all normal;
if it is determined that any one or a combination of the reticulocytes, the immature platelets and the large-volume platelets in the blood sample is abnormal, the processor further controls the blood sample supply portion to provide the blood sample to the mixing chamber, and controls the reagent supply portion to provide a second reaction reagent to the mixing chamber to prepare a second test sample in the mixing chamber, wherein the second reaction reagent contains no hemolytic agent; and the processor further controls the optical detection portion to obtain optical signals of the second test sample, obtains a second test result of the blood sample from the optical signals of the second test sample, and outputs the first test result, wherein the second test result comprises any one or a combination of a reticulocyte parameter, an immature platelet parameter and a large-volume platelet parameter of the second test sample.

23. The apparatus of claim 22, wherein the optical signals of the first test sample comprise forward-scattered light signals and fluorescence signals, wherein the processor further controls the blood sample supply portion to provide the blood sample to the mixing chamber and controls the reagent supply portion to provide the hemolytic agent and a first fluorescent agent to the mixing chamber to prepare the first test sample in the mixing chamber.

24. The apparatus of claim 22, wherein the processor determining whether any one or a combination of the reticulocytes, the immature platelets and the large-volume platelets in the blood sample is abnormal from the optical signals of the first test sample comprises:
the processor generating a scatter diagram corresponding to the first test sample from the optical signals of the first test sample;
the processor obtaining, on the scatter diagram of the first test sample, feature regions corresponding to the reticulocytes, the immature platelets and the large-volume platelets in the blood sample; and
the processor determining whether any one or a combination of the reticulocytes, the immature platelets and the large-volume platelets in the blood sample is abnormal based on scatter point information in the obtained feature regions.

25. The apparatus of claim 24, wherein the processor determining whether any one or a combination of the reticulocytes, the immature platelets and the large-volume platelets in the blood sample is abnormal based on scatter point information in the obtained feature regions comprises:
the processor counting first scatter points in the feature region corresponding to the reticulocytes in the blood sample;
the processor determining that the reticulocytes in the blood sample are normal if the count of the first scatter points is within a first threshold range;
the processor determining that the reticulocytes in the blood sample are abnormal if the count of the first scatter points is outside the first threshold range;
the processor counting second scatter points in the feature region corresponding to the immature platelets in the blood sample;
the processor determining that the immature platelets in the blood sample are normal if the count of the second scatter points is within a second threshold range;
the processor determining that the immature platelets in the blood sample are abnormal if the count of the second scatter points is outside the second threshold range;
the processor counting third scatter points in the feature region corresponding to the large-volume platelets in the blood sample; and
the processor determining that the large-volume platelets in the blood sample are normal if the count of the third scatter points is within a third threshold range;
the processor determining that the large-volume platelets in the blood sample are abnormal if the count of the third scatter points is outside the third threshold range.

26. The apparatus of claim 22, wherein the processor further generates a corresponding alarm prompt if it is determined that any one or a combination of the reticulocytes, the immature platelets and the large-volume platelets in the blood sample is abnormal.

27. The apparatus of claim 22, wherein the optical signals of the second test sample comprises forward-scattered light signals and fluorescence signals, wherein the processor further controls the blood sample supply portion to provide the blood sample to the mixing chamber and controls the reagent supply portion to provide a diluent and a second fluorescent agent to the mixing chamber to prepare the second test sample in the mixing chamber.

28. A blood analysis method, comprising:
determining whether a second measurement mode is enabled, wherein a blood analysis apparatus comprises a first measurement mode and the second measurement mode;
if the second measurement mode is enabled, preparing a second test sample, obtaining optical signals of the second test sample, and obtaining a second test result of a blood sample from the optical signals of the second test sample, wherein the second test result comprises any one or a combination of a reticulocyte parameter, an immature platelet parameter and a large-volume platelet parameter of the second test sample, the second test sample is obtained by treating the blood sample with at least a second reaction reagent, and the second reaction reagent contains no hemolytic agent;
if the second measurement mode is not enabled, obtaining optical signals of the first test sample in the first measurement mode, wherein the optical signals of the first test sample are used to determine a first test result of the blood sample, the first test result comprises a white blood cell count result and/or a white blood cell classification result, the first test sample is obtained by treating the blood sample with at least a first reaction reagent, and the first reaction reagent contains a hemolytic agent;
determining whether to enable the second measurement mode by determining whether any one or a combination of reticulocytes, immature platelets and large-volume platelets in the blood sample is abnormal from the optical signals of the first test sample; and if it is determined that any one or a combination of the reticulocytes, the immature platelets and the large-volume platelets in the blood sample is abnormal, it is determined that the second measurement mode should be enabled and the first test result is output; and if it is determined that the reticulocytes, the immature platelets and the large-volume platelets in the blood sample are all normal, it is determined that the second measurement mode should not be enabled and the first test result is output.

29. The method of claim 28, wherein the first test sample is obtained by treating the blood sample with the hemolytic agent and a first fluorescent agent, and the optical signals of the first test sample comprise forward-scattered light signals and fluorescence signals.

30. The method of claim 28, wherein determining whether any one or a combination of reticulocytes, immature platelets and large-volume platelets in the blood sample is abnormal from the optical signals of the first test sample comprises:
generating a scatter diagram corresponding to the first test sample from the optical signals of the first test sample;
obtaining, on the scatter diagram of the first test sample, feature regions corresponding to the reticulocytes, the immature platelets and the large-volume platelets in the blood sample; and
determining whether any one or a combination of the reticulocytes, the immature platelets and the large-volume platelets in the blood sample is abnormal based on scatter point information in the obtained feature regions.

31. The method of claim 30, wherein determining whether any one or a combination of the reticulocytes, the immature platelets and the large-volume platelets in the blood sample is abnormal according to scatter point information in the obtained feature regions comprises:
counting first scatter points in the feature region corresponding to the reticulocytes in the blood sample;
determining that the reticulocytes in the blood sample are normal if the count of the first scatter points is within a first threshold range;
determining that the reticulocytes in the blood sample are abnormal if the count of the first scatter points is outside the first threshold range;
counting second scatter points in the feature region corresponding to the immature platelets in the blood sample;
determining that the immature platelets in the blood sample are normal if the count of the second scatter points is within a second threshold range;
determining that the immature platelets in the blood sample are abnormal if the count of the second scatter points is outside the second threshold range;
counting third scatter points in the feature region corresponding to the large-volume platelets in the blood sample;
determining that the large-volume platelets in the blood sample are normal if the count of the third scatter points is within a third threshold range;
determining that the large-volume platelets in the blood sample are abnormal if the count of the third scatter points is outside the third threshold range.

32. The method of claim 28, further comprising: generating a corresponding alarm prompt if it is determined that any one or a combination of the reticulocytes, the immature platelets and the large-volume platelets in the blood sample is abnormal.

33. The method of claim 28, wherein the second test sample is obtained by treating the blood sample with a diluent and a second fluorescent agent, and the optical signal of the second test sample comprise forward-scattered light signals and fluorescence signals.
